(19) United States
(12) Patent Application Publication
(10) Pub. No.: US 2022/0258168 A1
(43) Pub. Date: Aug. 18, 2022

(54) DIAGNOSTIC CARTRIDGE FOR MICROFLUIDICS AND ON-SITE MOLECULAR DIAGNOSIS SYSTEM INCLUDING SAME

(71) Applicant: ADVANCED BIO VISION INC., Incheon (KR)

(72) Inventor: Dong Il KIM, Incheon (KR)

(73) Assignee: ADVANCED BIO VISION INC., Incheon (KR)

(21) Appl. No.: 17/625,316

(22) PCT Filed: Nov. 27, 2019
(86) PCT No.: PCT/KR2019/016443

§ 371 Date: Jan. 6, 2022

(30) Foreign Application Priority Data
Aug. 8, 2019 (KR) 10-2019-0096621

(51) Int. Cl. B01L 7/00; G01N 35/02

(52) U.S. Cl. CPC B01L 7/52; G01N 35/025; B01L 2200/0663; G01N 2035/00178; B01L 2300/1822; B01L 2300/1894; B01L 2300/0627

(57) ABSTRACT

The present invention relates to a diagnostic cartridge for microfluidics and an on-site molecular diagnosis system including same, wherein an infuser, an extraction chamber, and an amplification chamber are arrayed vertically to minimize the flow path of a fluid, and thus the extraction, amplification and analysis results of a nucleic acid can be detected in real time.

### TECHNICAL FIELD

The present invention relates to a diagnostic cartridge for microfluidic control and an on-site molecular diagnostic system including the same, and more specifically, a diagnostic cartridge for controlling microfluidics, in which an infuser, an extraction chamber and an amplification chamber are arranged in a vertical direction to implement a flow of fluid at the shortest distance, so as to enable extraction, amplification and detection of analyzed results of nucleic acid in real time, as well as an on-site molecular diagnosis system including the same.

### BACKGROUND ART

Recently, cases requiring more rapid diagnosis such as the emergence of new viruses are increased, therefore, a demand for genetic tests for the purpose of disease prevention is also increasing. As a solution for such requirement, a market of molecular diagnostics using a trace amount of nucleic acids is emerging as a promising field.

Molecular diagnosis refers to detection of genotypes or to determination of genetic mutations and biochemical changes in the body by measuring deoxyribonucleic acid (DNA), ribonucleic acid (RNA), protein or metabolites, etc. As a representative molecular diagnosis method, there is a method using a polymerase chain reaction (PCR) (hereinafter referred to as ‘PCR’). Using PCR/RT-PCR may check or confirm whether a specific DNA/RNA exists in a biological sample, so that it is widely used to diagnose infection by pathogenic microorganisms such as viruses.

In order to execute such PCR, a nucleic acid extraction step of extracting pure nucleic acid and removing substances that inhibit PCR reaction from a biological sample is required. The nucleic acid extraction process consists of multi-stages, and requires skilled technology to manipulate biological samples and nucleic acid extraction and, if it is done manually, may entail a problem of contamination due to an operator's error, so that most of the molecular diagnosis is implemented using automated nucleic acid extraction equipment.

Since a real-time quantitative PCR device must be equipped for PCR reaction and detection of reaction products, molecular diagnosis has been mainly conducted in large hospitals or specialized clinical laboratory institutions.

Meanwhile, POC (Point of Care) diagnosis technology, which accurately and quickly diagnoses a patient's disease regardless of time and place, is now attracting attention as a very important technology for evidence-based precision medicine. On the other hand, symptom-based on-site diagnosis wherein all infectious pathogens causing disease symptoms, in particular, cough, diarrhea, high fever and genital abnormalities, are examined in a quick time through a single test to identify the causative pathogen, followed by prescription of the optimal antibiotic and treatment, is a novel key technology for future precision medicine, and many studies are being added to develop the above technology. Such on-site diagnosis technology has an advantage of enabling quick and accurate diagnosis even by non-professionals in the field, such as a pregnancy test kit for confirming pregnancy and a blood glucose meter that can check blood sugar.

However, the current molecular diagnosis system takes more than 3 hours to confirm the results and must be used by experienced experts and, for POC molecular diagnosis required in the field, it is necessary to develop an automated and small device capable of fully automatically conducting a complicated nucleic acid extraction process and a real-time gene amplification. Further, it must be able to operate the device easily even by a non-professional manpower.

Through recent research and development, various automated systems and devices using the same have been developed to automate a whole process of nucleic acid extraction, PCR reaction and detection of reaction products, so that PCR can be easily used even without specialized technology.

However, existing devices are too expensive or take a long processing time. Further, it is difficult to perform various tests at once by the existing device.

Korean Patent Registration No. 10-1813870 discloses an automatic analysis device for molecular diagnosis, capable of conducting nucleic acid extraction, amplification, and detection in a continuous process, and enabling a genetic quantitative test and a qualitative test in combination.

However, in order to be suitable for on-site molecular diagnosis, there is a need for competitive and differentiated technologies that enable all-in-one automation of the entire processes including reduction of a turnaround time, sample preparation, nucleic acid amplification and detection, and multiplexing and continuous random tests, and can reduce inspection costs through lightweight system and process optimization.

### DISCLOSURE

### Technical Problem

The present invention is conceived to solve the above-described problems, and an object of the present invention is to provide a diagnostic cartridge capable of controlling fluid transfer by arranging an infuser, an extraction chamber and an amplification chamber in a vertical direction and desirably aligning grooves formed in the cartridge.

Another object of the present invention is to provide an on-site molecular diagnosis system that may be compactly configured because power such as a pump or an ultrasonic device is not used.

A further object of the present invention is to provide an on-site molecular diagnosis system capable of continuously and automatically conducting a series of processes including nucleic acid extraction, amplification and analysis while shortening the processing time.

### Technical Solution

The diagnostic cartridge according to the present invention for achieving the above objects may include: an infuser in which a plurality of chambers is configured in a cylindrical shape, a sample can be introduced into any one of the chambers, and a reagent is provided in any one or more chambers; an extraction chamber coupled to the infuser, which extracts nucleic acids through pulverization of a sample, cell destruction and washing of the sample; a waste chamber located below the extraction chamber and formed in a single cylindrical shape to collect waste; an amplification chamber located below the waste chamber, which includes a plurality of chambers and conducts nucleic acid amplification; and a rotatable cylindrical magnet holder coupled through a central portion of the infuser and an opening of the extraction chamber, wherein the infuser, the extraction chamber, the waste chamber and the amplification chamber are arranged in a vertical direction.

The magnet holder may be provided with a magnet at the lower end inside the cylinder to separate nucleic acids and waste products, and may rotate at a predetermined angle to move a fluid.

The extraction chamber may include: an upper edge portion of the extraction chamber coupled with a lower edge portion of the infuser; an extraction chamber wall inclined from the upper edge portion of the extraction chamber to form an opening in the center portion; and a lower edge portion of the extraction chamber formed at the end of the opening, wherein an extraction chamber hole is further provided to allow the fluid to vertically flow into the amplification chamber.

The waste chamber may be formed in a housing shape to accommodate the extraction chamber, may be coupled to the upper edge portion of the extraction chamber and have a space, by which a bottom surface of the waste chamber and a bottom surface of the extraction chamber are spaced apart from each other, and may be provided with a hose connection hole to generate a negative pressure at a lateral side of the housing.

The magnet holder, the extraction chamber and the amplification chamber, respectively, may have grooves formed at predetermined angles, and the grooves may be desirably aligned to control the flow of fluid.

As the magnet holder rotates at a rotation angle as shown in Equation 1 below with respect to the groove, the groove formed in the amplification chamber and the groove of the magnet holder may be aligned to move the fluid.

PCRₙ₊₁=PCRₙ+360/N [Equation 1]

(In Equation 1, PCRₙ refers to an angle between the groove and the amplification chamber located at n-th in the magnet holder rotational direction, N refers to the number of total amplification chambers, and each of PCRₙ and PCRₙ₊₁ is within ±5 degrees.)

When the magnet holder is rotated by the rotation angle of 10 to 20 degrees to the groove, the fluid may move from the extraction chamber to the waste chamber.

The on-site molecular diagnosis system according to the present invention for achieving the another object may include: a diagnostic cartridge including an infuser, an extraction chamber, a waste chamber, an amplification chamber and a magnet holder; a nucleic acid extraction module for extracting nucleic acids by mixing and transferring reagents and samples contained in the cartridge; a temperature control module for amplifying the nucleic acid by heating and cooling the nucleic acid extracted from the nucleic acid extraction module; an optical module for detecting a fluorescent signal generated from the amplified nucleic acid; and an integrated control board for controlling operations of the nucleic acid extraction module, the temperature control module and the optical module, wherein the diagnostic cartridge includes the infuser, the extraction chamber, the waste chamber and the amplification chamber arranged in a vertical direction.

The nucleic acid extraction module may include a plurality of motors, syringes and negative pressure valves.

The motor may include a first motor to a fifth motor, wherein the first motor is coupled to a magnetic rod to transport the magnetic rod and secure the magnet holder, the second motor is coupled to a pumping rod to conduct pumping inside the infuser, the third motor transfers the negative pressure valve, the fourth motor transfers the syringe to form a negative pressure, and the fifth motor rotates the diagnostic cartridge.

The temperature control module may include a Peltier element, a radiating fin, a cooling fan and an amplification chamber fixing block.

The temperature control module may be coupled to the diagnostic cartridge through the amplification chamber fixing block, and may rotate along with rotation of the diagnostic cartridge.

The optical module may include one or more fluorescent sensors.

### Advantageous Effects

The diagnostic cartridge according to the present invention can realize the movement of fluid in the shortest distance by arranging the infuser, the extraction chamber, and the amplification chamber in a vertical direction, and has an advantage of easily controlling the transfer of fluid by aligning the grooves formed inside the cartridge in a suitable manner.

Further, power such as a pump or ultrasonic device is not used and fluid transfer is possible only by an arrangement of the chambers in the cartridge, thereby minimizing the power. Further, there is an advantage of compactly configuring the system to be suitable for on-site diagnosis.

Further, there is an advantage of significantly reducing the risk of contamination by minimizing a path through which the fluid flows.

Further, according to the present invention, a series of processes including nucleic acid extraction, amplification and analysis may be continuously and automatically performed, while reducing a processing time.

### DESCRIPTION OF DRAWINGS

FIG. 1 is a perspective view of an on-site molecular diagnosis system according to an embodiment of the present invention.

FIG. 2 is a front view (a) and a side view (b) of the on-site molecular diagnosis system according to an embodiment of the present invention.

FIG. 3 is an exploded perspective view of a diagnostic cartridge according to an embodiment of the present invention.

FIG. 4 is a side view (a) and a perspective view (b) of the diagnostic cartridge according to an embodiment of the present invention.

FIG. 5 is a plan view (a) and a perspective view (b) of the diagnostic cartridge according to an embodiment of the present invention.

FIG. 6 is an exploded perspective view of a temperature control module according to an embodiment of the present invention.

FIG. 7 is a plan view (a), a perspective view (b), and side views (c, d) of the temperature control module according to an embodiment of the present invention.

FIG. 8 is a plan view (a) and a perspective view (b) illustrating a configuration in which a temperature control module and a diagnostic cartridge are combined according to an embodiment of the present invention.

FIG. 9 is a side view of the combination of the temperature control module and the diagnostic cartridge according to an embodiment of the present invention.

FIG. 10 shows multiple chambers of the amplification chamber according to an embodiment of the present invention.

FIG. 11 shows multiple chambers of the infuser according to an embodiment of the present invention.

### DETAILED DESCRIPTION OF PREFERRED EMBODIMENTS OF INVENTION

Advantages and features of the present invention, and a method of achieving the same will become apparent with reference to the embodiments described below in detail together with the accompanying drawings. However, the present invention is not limited to the embodiments disclosed below but will be implemented in a variety of different forms. In fact, these embodiments are used only to make the disclosure of the present invention complete and are provided to completely inform the scope of the invention to those who have the general knowledge in the technical field to which the present invention pertains. Therefore, the present invention is only defined by the scope of the claims. Further, the same reference numerals refer to the same elements throughout the specification.

In addition, when a component is referred to as being “connected” or “coupled” to another component, it means that the component can be logically or physically connected or combined to the another component. In other words, it should be understood that a component may be directly connected or coupled to another component, a further component may exist therebetween, or a component may be indirectly connected or coupled to another component.

Hereinafter, a detailed description will be given of a diagnostic cartridge for microfluidic control and an on-site molecular diagnostic system including the same according to preferred embodiments of the present invention with reference to the accompanying drawings.

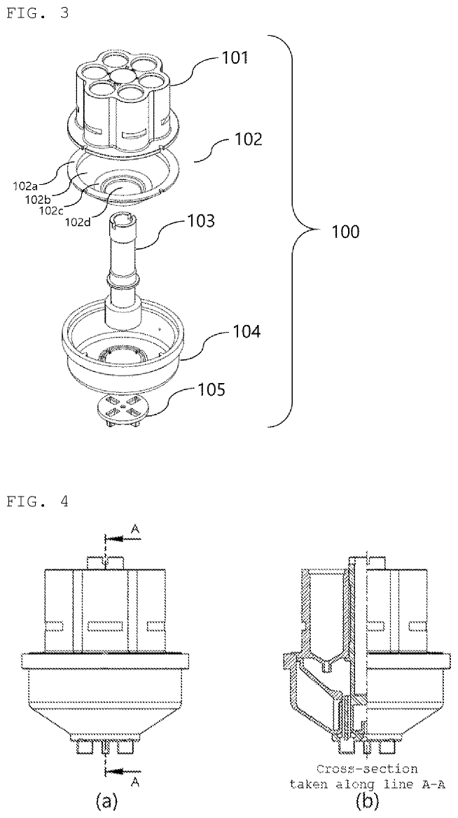

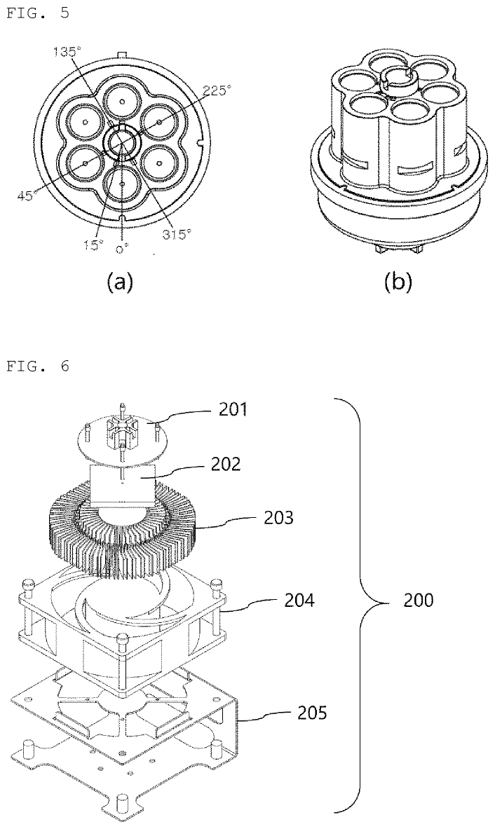

FIG. 1 is a perspective view of an on-site molecular diagnosis system according to an embodiment of the present invention, and FIG. 3 is an exploded perspective view of a diagnostic cartridge according to an embodiment of the present invention.

Referring to FIG. 3, the diagnostic cartridge according to the present invention may include an infuser, an extraction chamber, a waste chamber, a magnet holder and an amplification chamber, wherein the infuser, extraction chamber, waste chamber and amplification chamber are arranged in a vertical direction.

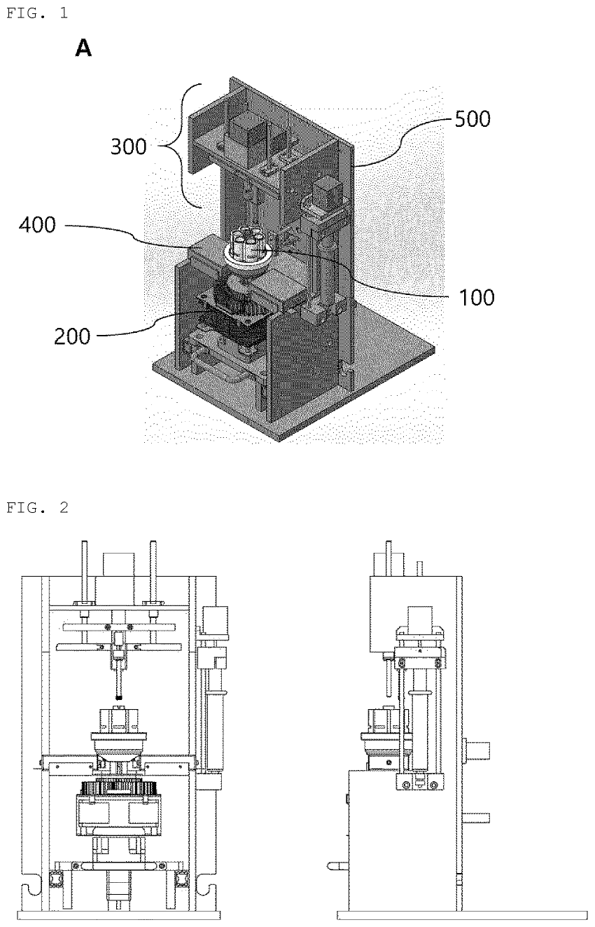

Further, referring to FIGS. 1 to 2, the present invention provides an on-site molecular diagnostic system using a diagnostic cartridge, characterized in that the above-described diagnostic cartridge, a nucleic acid extraction module, a temperature control module, an optical module and an integrated control board are included, whereby pre-treatment, nucleic acid extraction and purification, nucleic acid amplification, and fluorescence detection may be performed in a lump on a sample (specimen) put into the diagnostic cartridge.

Hereinafter, the diagnostic cartridge will be described in more detail.

Diagnostic Cartridge

Referring to FIG. 3, the diagnostic cartridge 100 of the present invention may include an infuser 101, an extraction chamber 102, a magnet holder 103, a waste chamber 104, and an amplification chamber 105.

The infuser 101 may be provided with a sample (specimen) and a reagent, wherein the sample (specimen) is introduced from the outside using a syringe or the like, and the reagent is provided in the infuser or introduced from the outside. In the case of inputting the reagent from the outside, an experimenter may directly introduce the reagent or a separate automated device may be used.

The reagent provided in the infuser 101 may include, for example, magnetic particles, a lysate for destroying and lysing cells in the sample (specimen), a purification solution for purifying nucleic acids in the destroyed cells, and/or an eluate for separating nucleic acids, but is not limited thereto.

The sample (specimen) is a bio-sample requiring pretreatment, and may be selected from saliva, sputum, tissue, blood, urine, feces, spinal fluid, mucus, etc., but is not limited thereto.

As shown in FIG. 11, the infuser 101 may have a plurality of chambers formed in a cylindrical shape and may include a hollow portion in which the magnet holder 103 is coupled in the center. The number of chambers is not limited but is preferably 6 to 8.

Referring to FIG. 4(b), an infuser hole through which fluid can move to the extraction chamber 102 is provided below any one or more of the multiple chambers to form the infuser 101 and, in order to facilitate the flow of fluid, a wall connected to an infuser hole may be formed to be inclined. The infuser hole may be accommodated into the extraction chamber 102 so as to be shielded from the outside.

The extraction chamber 102 may be coupled to the infuser 101 and serve to extract nucleic acids through pulverization, cell destruction and washing of the sample.

The extraction chamber 102 may include: an upper edge portion 102a coupled with a lower edge portion of the infuser 101; an extraction chamber wall 102b inclined from the upper edge portion 102a in order to form an opening in the center portion; and a lower edge portion 102c formed at the end of the opening, and may further be provided with an extraction chamber hole 102d through which the fluid vertically flows to the amplification chamber 105.

The magnet holder 103 is a rotatable cylindrical structure that is coupled through the central portion of the infuser 101 and the opening of the extraction chamber 102, and may have a magnet inside the cylinder to separate nucleic acids and waste. Further, the fluid may be moved by rotating the magnet holder at a predetermined angle.

The magnet holder 103, the extraction chamber 102, and the amplification chamber 105, respectively, may have grooves formed at predetermined angles, and the grooves may be suitably aligned to control the flow of fluid.

As the magnet holder 103 rotates at a rotation angle as shown in Equation 1 below with respect to the groove, the groove formed in the amplification chamber and the groove of the magnet holder are aligned so that the amplification chamber 105 can move the fluid without passing through the waste chamber 104.

PCRₙ₊₁=PCRₙ+360/N [Equation 1]

(In Equation 1, PCRₙ refers to the angle between the groove and the amplification chamber located n-th in the magnet holder rotating direction, and N refers to the number of total amplification chambers, and each of PCRₙ and PCRₙ₊₁ is within ±5 degrees.)

For example, if the total number of amplification chambers is 4 and the first amplification chamber in the direction of rotating the magnet holder exists at an angle of 45 degrees to the groove, the second chamber is positioned at 45 degrees+360/4 (=90), that is, at 135 degrees from the groove.

When the magnet holder 103 is rotated by the rotation angle of 10 to 20 degrees to the groove, the fluid may move from the extraction chamber 102 to the waste chamber 104. The rotation angle is preferably 12 to 17 degrees, more preferably 15 degrees.

The waste chamber 104 may be located under the extraction chamber 102 and formed in a single cylindrical shape to collect waste. The waste may be a waste liquid generated while pre-treating a sample (specimen) in the extraction process of a nucleic acid, and may include the solution, the purification solution, the eluate, etc. provided in the infuser 101.

The waste chamber 104 may be formed in a housing shape to accommodate the extraction chamber 102, may be coupled to the upper edge portion 102a of the extraction chamber and have a space, by which a bottom surface of the waste chamber 104 and a bottom surface of the extraction chamber are spaced apart from each other, and may be provided with a hose connection hole to generate a negative pressure at a lateral side of the housing.

By forming a negative pressure in the waste chamber 104, it is possible to facilitate the movement of the fluid more smoothly.

The amplification chamber 105 may be located under the waste chamber 104, and may include a plurality of chambers to perform nucleic acid amplification. The amplification reagent may be provided in the amplification chamber or may be provided in the infuser chamber.

According to an embodiment of the present invention, the infuser, the extraction chamber, the waste chamber and the amplification chamber may be arranged in a vertical direction. With such vertical arrangement of the infuser, the extraction chamber, the waste chamber and the amplification chamber, a flow path of the fluid may have the shortest distance while being shielded from the outside, thereby accomplishing the advantages in that the system becomes compact and the sample (a mixture of the sample (specimen) and a reagent) is protected from contamination.

Hereinafter, the on-site molecular diagnosis system will be described in more detail.

On-Site Molecular Diagnosis System

Referring to FIG. 1, the on-site molecular diagnosis system A of the present invention may include a diagnostic cartridge 100, a nucleic acid extraction module 300, a temperature control module 200, an optical module 400, and an integrated control board 500.

The diagnostic cartridge 100 is as described above.

The nucleic acid extraction module 300 may extract nucleic acids by mixing a reagent and a sample (specimen) contained in the cartridge and then transferring the mixture to the module.

The nucleic acid extraction module 300 may include a plurality of motors, syringes and negative pressure valves.

The motor may include first to fifth motors.

The first motor is located at an upper end of the diagnostic cartridge 100, is coupled with a magnetic rod to move the magnetic rod up and down, and plays roles of drawing the magnet rod in or out a hollow portion formed inside the diagnostic cartridge, that is, the center portion of the infuser 101, while fixing the magnet holder. When the magnetic rod is inserted, magnetic particles bound to the nucleic acid may be held using a magnetic force in order to extract the nucleic acid during pretreatment of the sample (specimen). When the magnetic rod is inserted into the cartridge, the magnet holder 103 may be fixed.

The second motor is located on a lateral side of the first motor, is coupled with a pumping rod to move up and down, and may conduct pumping in the infuser 101. More specifically, in order to transfer reagents required in the pretreatment of the sample (specimen) to the extraction chamber, it may be pumped using a second motor. A piston may be further included to facilitate pumping.

The third and fourth motors are for generating a negative pressure, wherein the third motor transports a negative pressure valve and the fourth motor transports a syringe to generate the negative pressure.

The third motor may be connected to a hose connection hole for generating a negative pressure formed in a side wall of the housing of the waste chamber 104.

The fourth motor may be located on the lateral side of the diagnostic cartridge 100 and connected to the syringe, and may generate a negative pressure while rising, and further generate a normal pressure while falling. The syringe may be connected to a hose for generating a negative pressure.

The fifth motor may be located at the lower end of the temperature control module 200 and may rotate the diagnostic cartridge at a predetermined angle.

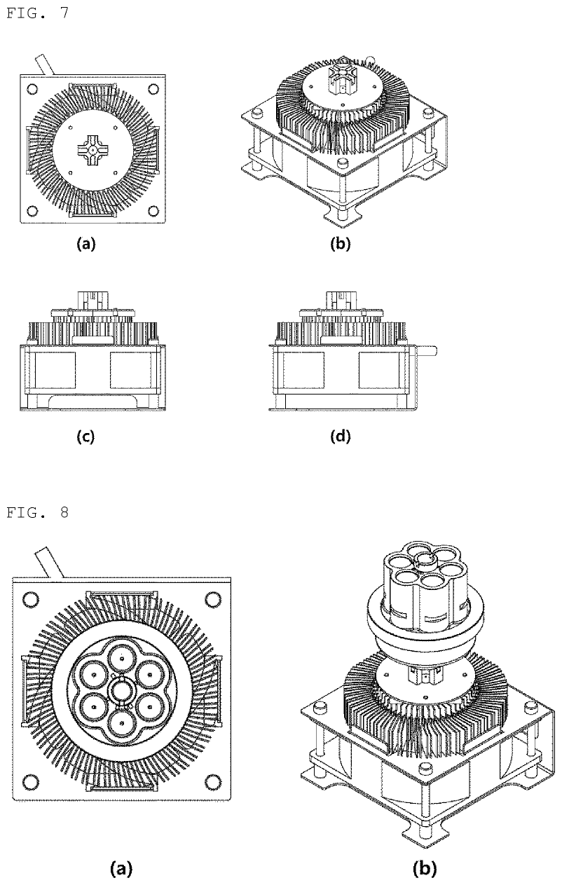

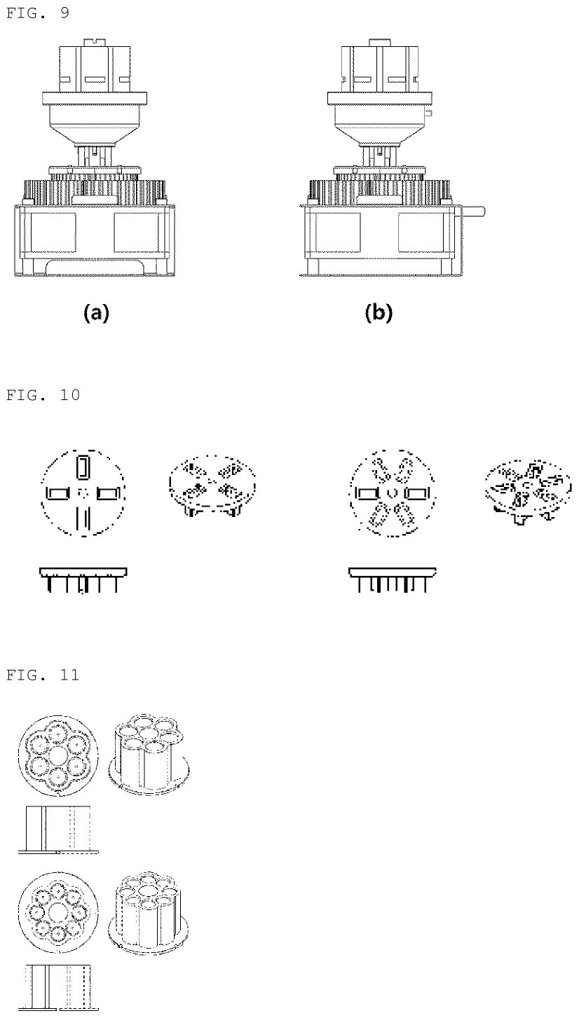

FIGS. 6 to 9 illustrate the temperature control module 200 according to an embodiment of the present invention.

The temperature control module 200 may include a Peltier element, a radiating fin, a cooling fan, and an amplification chamber fixing block, and may amplify the nucleic acid extracted in the nucleic acid extraction module 300 by heating and cooling the nucleic acid.

The temperature control module 200 may be coupled to the diagnostic cartridge through the amplification chamber fixing block, and may rotate along with rotation of the diagnostic cartridge 100. The Peltier element may be used as a heat source, and the amplification chamfer fixing block may secure the amplification chamber and transfer heat at the same time.

The temperature control module 200 may further include one or more temperature sensors.

The optical module 400 may detect a fluorescence signal generated from the amplified nucleic acid, and may include one or more fluorescence sensors. Specifically, the optical module may be configured in the form of multiple sensors so as to detect light in different wavelengths.

The fluorescence sensor may include a light-emitting part and a light-receiving part, wherein fluorescence reflected from the light irradiated from the light-emitting part to the amplification chamber is received and then converted into a fluorescent signal.

The integrated control board 500 may control driving (or operation) of the nucleic acid extraction module 300, the temperature control module 200 and the optical module 400. Specifically, the above operation may be controlled in an integrated manner using a communication method, and the control board may be connected to a separate PC to configure a user's display screen.

As described above, the diagnostic cartridge for controlling microfluidics and the on-site molecular diagnostic system according to the present invention may perform molecular diagnosis while automatically implementing a lysis process, a washing process, an elusion process, an amplification (PCR) process and a detection process.

For example, a diagnostic cartridge in which a sample (specimen) is introduced may be positioned to be coupled with the amplification chamber fixing block of the temperature control module. One of the infusion chambers, into which the sample (specimen) is introduced, may be located under the pumping rod connected to the second motor. The sample (specimen) may be pushed down into the extraction chamber through piston motion of the pumping rod. Next, when the fifth motor rotates about 60 degrees in a clockwise direction, both of the diagnostic cartridge and the temperature control module may rotate by 60 degrees so that a chamber in which magnetic particles are introduced may be located under the pumping rod connected to the second motor. Thereafter, the pumping rod may push gain the magnetic particles down into the extraction chamber through piston motion and, when the fifth motor rotates again clockwise by about 60 degrees, the chamber in which the lysate is introduced may be located under the pumping rod. When the pumping rod takes the piston motion again, the lysate may flow into the extraction chamber, and the sample (specimen), magnetic particles and lysate may be mixed in the extraction chamber, thereby separating nucleic acids from the cells. Next, when the magnetic rod is brought into the magnet holder by the first motor, magnetic particles adsorbed with nucleic acid may be adhered to the magnetic rod. At this time, if the magnet holder is rotated by 15 degrees, waste mixed with other cell fluids and lysate may flow into the waste chamber except for the magnetic particles adsorbed with nucleic acid. In this case, the fourth motor and the syringe may be driven to create a negative pressure to facilitate the flow of fluid. When the fifth motor rotates clockwise by about 60 degrees, the chamber in which the purification solution is introduced may be located under the pumping rod, and the purification solution may flow into the extraction chamber through a piston motion of the pumping rod. When the magnetic rod is drawn out of the magnet holder using the first motor, the purification solution and the magnetic particles adsorbed with nucleic acid may be mixed and purified. The purification process may be repeated once or twice to remain the nucleic acid necessary for detection. After the purification process is completed and when the magnetic rod is inserted into the magnet holder using the first motor, magnetic particles with only pure target nucleic acid adsorbed thereto may be adhered to the magnetic rod. At this time, when the magnet holder is rotated by about 15 degrees, the purification solution containing foreign substances except for the magnetic particles containing pure target nucleic acid adsorbed thereto may flow into the waste chamber. In this case, the fourth motor and the syringe may be driven to create a negative pressure to facilitate the flow of fluid.

After purification, when the fifth motor rotates about 60 degrees, the chamber into which the eluate is introduced may be located under the pumping rod.

Then, when the eluate flows into the extraction chamber through a piston motion of the pumping rod, the nucleic acids adsorbed on the magnetic particles may be separated from the magnetic particles. While the magnet holder is rotated by about 90 degrees from the amplification chamber located first with respect to the groove by the first motor, nucleic acid in the extraction chamber is flowed into the amplification chamber containing an amplification reagent. Thereafter, the nucleic acid may be amplified in the temperature control module through heating and cooling, followed by detecting fluorescence signals in specific wavelengths using the optical module.

In the lysis process, unnecessary vibration may be eliminated by dissolving the sample (specimen) with only a reagent without using a separate ultrasonic device for cell pulverization, thereby achieving advantages of reducing errors and enabling the system compact.

Further, the diagnostic cartridge according to the present invention may realize a movement of fluid in the shortest distance by arranging the infuser, the extraction chamber and the amplification chamber in a vertical direction, and the transfer of the fluid may be easily controlled by properly aligning the grooves formed inside the cartridge. In addition, there is an advantage of significantly reducing the risk of contamination and shortening a processing time.

Although the embodiments of the present invention have been described with reference to the accompanying drawings, the present invention is not limited to the above embodiments but may be manufactured in various different forms. Further, those skilled in the art to which the present invention pertains will understand that the present invention can be implemented in other specific forms without changing the technical spirit or essential features of the present invention. Therefore, it should be understood that the embodiments described above are illustrative and non-limiting in all respects.

### DESCRIPTION OF REFERENCE NUMERALS

100: Diagnostic cartridge
101: Infuser, 102: Extraction chamber 103: Magnet holder 104: Waste chamber
105: Amplification chamber
102a: Upper edge portion of extraction chamber 102b: Extraction chamber wall
102c: Lower edge portion of extraction chamber 102d: Extraction chamber hole
200: Temperature control module
201: Amplification chamber fixing block 202: Peltier element 203: Radiating fin
204: Fan 205: Fan support
300: Nucleic acid extraction module
400: Optical module
500: Integrated control board
A: On-site molecular diagnosis system

1. A diagnostic cartridge, comprising:
an infuser in which a plurality of chambers is configured in a cylindrical shape, a sample can be introduced into any one of the chambers and a reagent is provided in any one or more chambers;
an extraction chamber coupled to the infuser, and extracting nucleic acids through pulverization of a sample, cell destruction and washing of the sample;
a waste chamber located below the extraction chamber and formed in a single cylindrical shape to collect waste;
an amplification chamber located below the waste chamber, which includes a plurality of chambers and conducts nucleic acid amplification; and
a rotatable cylindrical magnet holder coupled through a central portion of the infuser and an opening of the extraction chamber,
wherein the infuser, the extraction chamber, the waste chamber and the amplification chamber are arranged in a vertical direction.

2. The diagnostic cartridge according to claim 1, wherein the magnet holder is provided with a magnet at a lower end inside the cylinder to separate nucleic acids and waste products, and rotates at a predetermined angle to move a fluid.

3. The diagnostic cartridge according to claim 1, wherein the extraction chamber includes:
an upper edge portion of the extraction chamber coupled with a lower edge portion of the infuser;
an extraction chamber wall inclined from the upper edge portion of the extraction chamber to form an opening in the center portion; and
a lower edge portion of the extraction chamber formed at an end of the opening,
wherein an extraction chamber hole is further provided to allow the fluid to vertically flow into the amplification chamber.

4. The diagnostic cartridge according to claim 1, wherein the waste chamber is formed in a housing shape to accommodate the extraction chamber, is coupled to an upper edge portion of the extraction chamber and has a space, by which a bottom surface of the waste chamber and a bottom surface of the extraction chamber are spaced apart from each other, and is provided with a hose connection hole to generate a negative pressure at a lateral side of the housing.

5. The diagnostic cartridge according to claim 1, wherein the magnet holder, the extraction chamber and the amplification chamber, respectively, have grooves formed at predetermined angles, and the grooves are aligned to control the flow of fluid.

6. The diagnostic cartridge according to claim 5, wherein, when the magnet holder rotates at a rotation angle as shown in Equation 1 below with respect to the groove, the groove formed in the amplification chamber and the groove of the magnet holder are aligned to move the fluid:
PCRₙ₊₁=PCRₙ+360/N [Equation 1]
(In Equation 1, PCRₙ refers to an angle between the groove and the amplification chamber located at n-th in the magnet holder rotational direction, N refers to the number of total amplification chambers, and each of PCRₙ and PCRₙ₊₁ is within ±5 degrees.)

7. The diagnostic cartridge according to claim 5, wherein, when the magnet holder is rotated by a rotation angle of 10 to 20 degrees to the groove, the fluid moves from the extraction chamber to the waste chamber.

8. An on-site molecular diagnosis system, comprising:
a diagnostic cartridge including an infuser, an extraction chamber, a waste chamber, an amplification chamber and a magnet holder;
a nucleic acid extraction module for extracting nucleic acids by mixing and transferring reagents and samples contained in the cartridge;
a temperature control module for amplifying the nucleic acid by heating and cooling the nucleic acid extracted from the nucleic acid extraction module;
an optical module for detecting a fluorescent signal generated from the amplified nucleic acid; and
an integrated control board for controlling operation of the nucleic acid extraction module, the temperature control module and the optical module,
wherein the diagnostic cartridge includes the infuser, the extraction chamber, the waste chamber and the amplification chamber arranged in a vertical direction.

9. The system according to claim 8, wherein the nucleic acid extraction module includes a plurality of motors, syringes and negative pressure valves.

10. The system according to claim 9, wherein the motor includes a first motor to a fifth motor,
wherein the first motor is coupled to a magnetic rod to transport the magnetic rod and secure the magnet holder,
the second motor is coupled to a pumping rod to conduct pumping inside the infuser,
the third motor transfers the negative pressure valve,
the fourth motor transfers the syringe to form a negative pressure, and
the fifth motor rotates the diagnostic cartridge.

11. The system according to claim 8, wherein the temperature control module includes a Peltier element, a radiating fin, a cooling fan and an amplification chamber fixing block.

12. The system according to claim 11, wherein the temperature control module is coupled to the diagnostic cartridge through the amplification chamber fixing block, and rotates along with rotation of the diagnostic cartridge.

13. The system according to claim 8, wherein the optical module includes one or more fluorescent sensors.
